(19) United States
(12) Patent Application Publication
(10) Pub. No.: US 2010/0145340 A1
(43) Pub. Date: Jun. 10, 2010

(54) Introducer Tool for Bone Measurement

(75) Inventors: Christopher U. Phan, San Leandro, CA (US); Gary A. Schneiderman, Sacramento, CA (US)

Correspondence Address: MEDTRONIC;Attn: Noreen Johnson - IP Legal Department, 2600 Sofamor Danek Drive, MEMPHIS, TN, 38132, US

(73) Assignee: KYPHON SARL, Neuchatel (CH)

(21) Appl. No.: 12/328,873

(22) Filed: Dec. 5, 2008

(51) Int. Cl. A61B 17/00; A61B 17/58; A61M 29/00

(52) U.S. Cl. 606/79; 606/96; 606/104; 604/96.01; 606/102

(57) ABSTRACT

A system for performing a medical procedure includes an introducer and a guide pin that include features that coordinate to indicate the distance the guide pin is extended beyond the tip of the introducer when both are deployed at a target location. By incorporating measurement features into the guide pin/introducer combination, accurate measurement capabilities are provided without requiring dedicated measurement tools.

### FIELD OF THE INVENTION

The invention relates to a system and method for performing a surgical procedure, and in particular, to a medical kit or system that includes an introducer/guide pin system that enables rapid and accurate depth measurement.

### BACKGROUND OF THE INVENTION

A minimally invasive procedure is a medical procedure that is performed through the skin or an anatomical opening. In contrast to an open procedure for the same purpose, a minimally invasive procedure will generally be less traumatic to the patient and result in a reduced recovery period.

However, there are numerous challenges that minimally invasive procedures present. For example, minimally invasive procedures are typically more time-consuming than their open procedure analogues due to the challenges of working within a constrained operative pathway. In addition, without direct visual feedback into the operative location, accurately selecting, sizing, placing, and/or applying minimally invasive surgical instruments and/or treatment materials/devices can be difficult.

For example, in a conventional open posterior fixation procedure (typically performed to aid spinal fusion), screws and rods are fastened into pedicles or across facet joints of a spinal column to immobilize two or more vertebrae. An open procedure allows the surgeon to select and place appropriately sized pedicle or facet screws based on unobstructed observation of the relevant vertebral structure(s).

However, performing a minimally invasive posterior fixation to aid spinal fusion procedure means that the surgeon must select and place the fixation hardware based on more indirect assessments, such as x-ray fluoroscopy or percutaneous measurement tools. Unfortunately, fluoroscopy does not provide a highly precise indication of dimensional measurements. To obtain greater measurement accuracy, dedicated percutaneous measurement tools can be used, but the use of such tools can increase procedure cost (due to extra tools required) and duration/complexity (due to extra procedural steps).

Accordingly, it is desirable to provide minimally invasive surgical tools that enable efficient and accurate measurement of internal anatomical regions is desired.

### SUMMARY OF THE INVENTION

By incorporating measurement indicators into the guide pin and introducer used to define and create the access path for a minimally invasive procedure, accurate and efficient depth measurements can be generated for appropriate sizing, selecting, and/or usage of devices and tools used in the procedure. Eliminating the need for supplemental measurement instruments/steps while still providing precise measurement data can not only enhance the safety and likelihood of success for the procedure, but can also beneficially reduce procedure duration and cost.

In one embodiment, a system for performing a medical procedure includes an introducer for creating a passage through tissue and a guide pin sized to pass through a lumen in the introducer. The introducer includes an elongate shaft with a distal tip and an indicator element. The guide pin includes a marker element, such that when the guide pin is within the introducer lumen, the indicator element in cooperation with the marker element indicates the distance the guide pin extends beyond the distal tip of the introducer.

In various embodiments, the marker element can be a single or multiple marks or features on the guide pin that can be compared to/detected by the indicator element on the introducer. In one embodiment, the indicator element includes a window to the introducer lumen and a series of tick marks adjacent to the window. The position of the marks/features on the guide pin relative to the series of tick marks on the indicator element can then be used to determine the position of the guide pin relative to the introducer.

In other embodiments, the indicator element can be a sensor, detector, or mechanism that responds to the marker element on the guide pin. The output of the sensor, detector, or mechanism can then be used to determine the position of the guide pin relative to the introducer.

In one embodiment, the system can include a bone screw driver tool, and the measurement capabilities of the introducer/guide pin combination can be used to select an appropriately-sized bone screw for deployment using the bone screw driver. Such an embodiment can further include a drill bit with optional depth indicators and/or removable depth stop that can be used in the drilling of a pilot hole for the installation of the bone screw. The depth indicators/depth stop can be used in conjunction with a working cannula to ensure that a pilot hole of appropriate depth is created, based on the measurement taken by the introducer/guide pin combination.

In another embodiment, the system can include inflatable bone tamps or other bone void creation devices of varying sizes. The measurement capabilities of the introducer/guide pin combination can be used to select an appropriately sized inflatable bone tamp (e.g., balloon length) or other bone void creation device to create an optimally-sized void within a vertebra for subsequent filling with bone filler material, as in balloon kyphoplasty.

In another embodiment, the system can include a cannulated needle with internal stylet for creating an initial trajectory into the subject body and to the internal anatomical target. The internal stylet can then be removed from the needle lumen, which can then be used to guide the guide pin to the target, at which point the guide pin can be driven to the desired depth into the target and the needle removed. The introducer (inserted into the working cannula) can then be placed over the guide pin and placed against the anatomical target to enable measurement of the guide pin depth within the target.

In another embodiment, the system can include an introducer stylet that can be removably placed within, and substantially fill, the introducer lumen. With the introducer stylet in place, the introducer can be driven directly into the subject body and to the surface of the internal anatomical target. The introducer stylet can then be removed and replaced with the guide pin, and can then be driven to the desired depth into the target, with the depth measurement being indicated by the indicator element/marker element combination.

As will be realized by those of skilled in the art, many different embodiments of an introducer/guide pin device, systems, kits, and/or methods of using an introducer/guide pin device according to the present invention are possible. Additional uses, advantages, and features of the invention are set forth in the illustrative embodiments discussed in the detailed description herein and will become more apparent to those skilled in the art upon examination of the following.

### BRIEF DESCRIPTION OF THE DRAWINGS

FIG. 1A shows a system for performing a medical procedure that includes an introducer and guide pin that provide integrated measurement capabilities.

FIGS. 1B-1L show exemplary usages of the system of FIG. 1A to perform medical procedures guided by measurements taken using the introducer/guide pin combination.

FIG. 2 is a flow diagram for a medical procedure using the system of FIG. 1A.

FIGS. 3A-3K show an exemplary use of the system of FIG. 1A to place bone screws in a posterior fixation to aid spinal fusion procedure.

FIGS. 4A-4C show an exemplary use of the system of FIG. 1A to perform a kyphoplasty or vertebroplasty procedure.

### DETAILED DESCRIPTION

By incorporating measurement indicia into the guide pin and introducer used to create the access path for a minimally invasive procedure, accurate and efficient depth measurements can be generated for use in selecting appropriately-sized tools/devices for use in the procedure.

### Terminology

In the context of the present disclosure, “anterior” refers to in front of the spinal column (ventral); “posterior” refers to behind the column (dorsal). The terms “proximal” and “distal” are defined with respect to the surgeon performing the operation. Thus, with respect to components used by the surgeon, the end of a component that is normally held by or is closest to the surgeon during use is considered to be proximal, and the end of a component that is placed into a patient or is furthest from the surgeon during use is considered to be distal.

### System

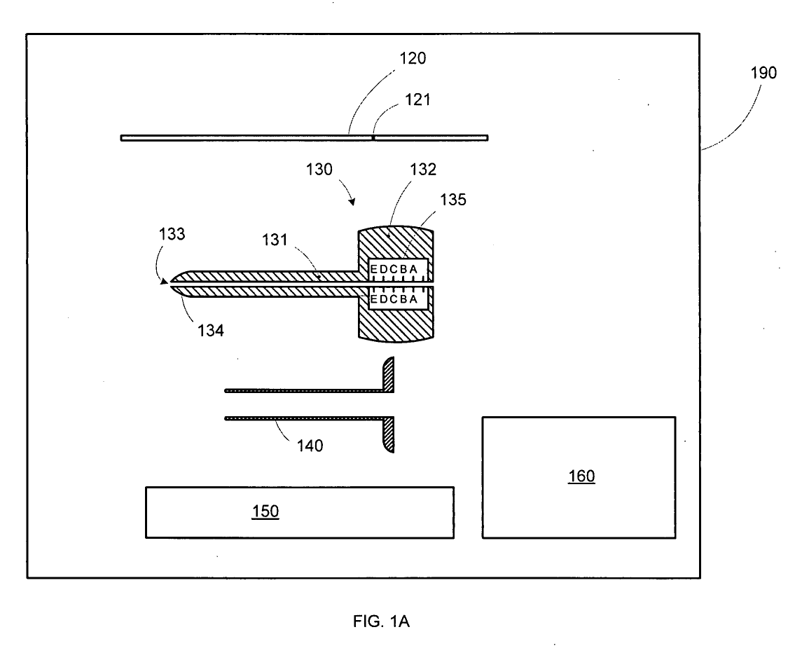

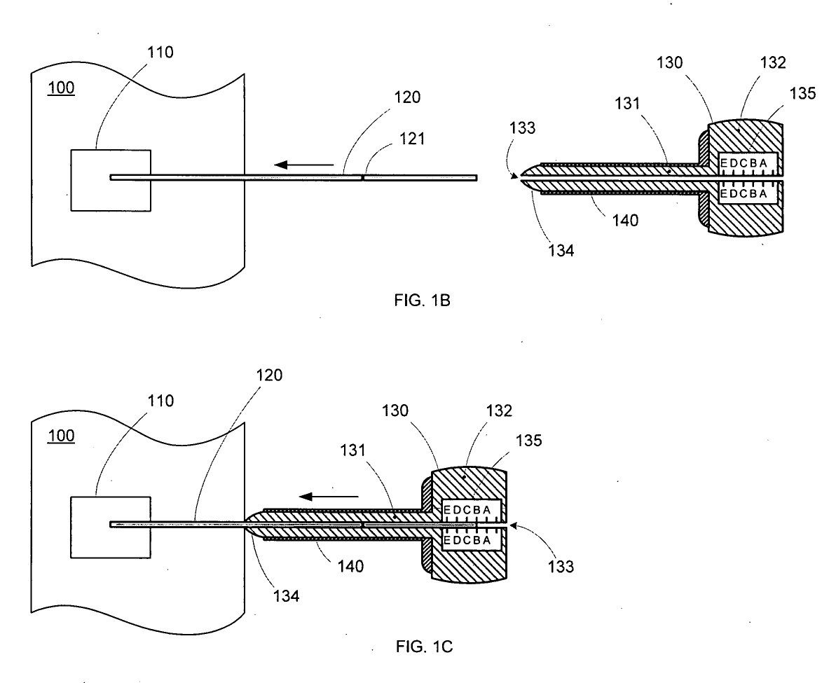

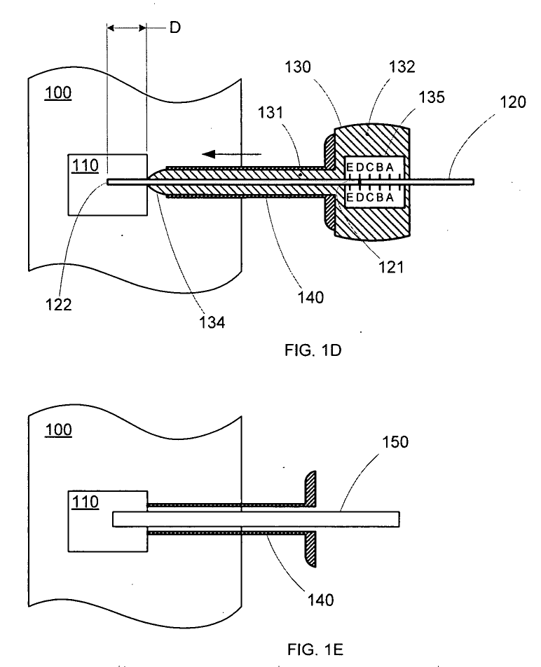

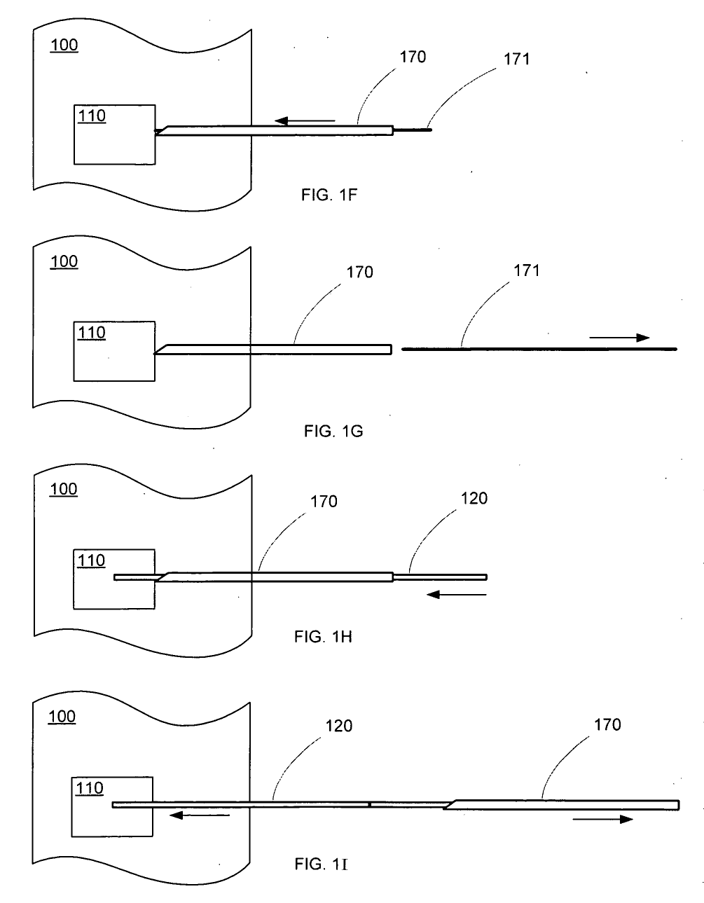

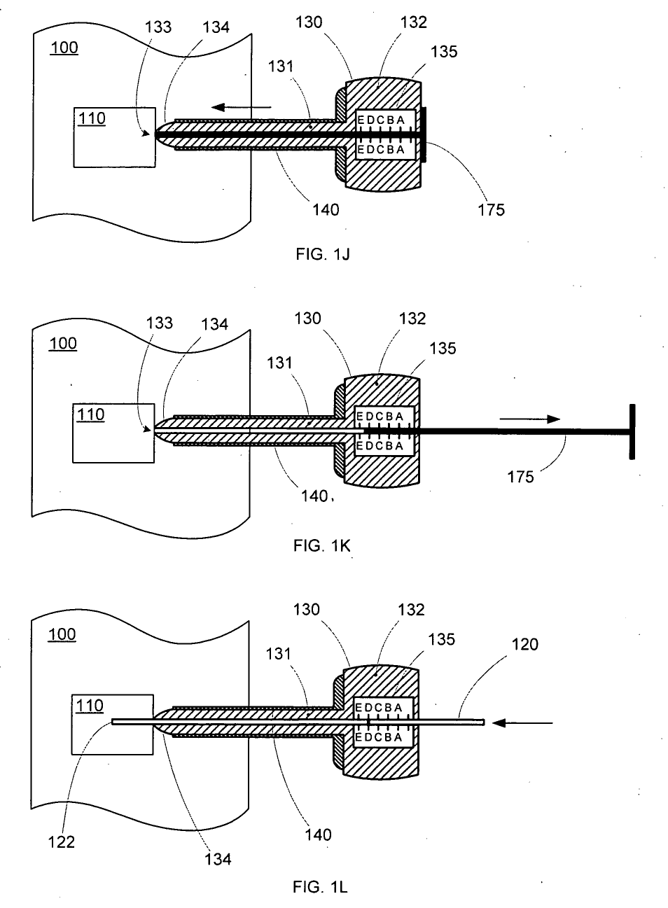

In one embodiment, a system 190 of functional instruments shown in FIG. 1A can be used to penetrate tissue and gain access to an internal anatomical target to perform a medical procedure. System 190 includes a guide pin 120, an introducer 130 (sometimes referred to as an obturator), a working cannula 140, optional additional tools 150, and optional instructions for use 160. System 190 can be provided as a prepackaged kit as sterile, wrapped assembly. Note that in other embodiments, the functional instruments of system 190 can be provided individually or in various combinations.

As described in greater detail below, guide pin 120 and introducer 130 are used to place working cannula 140 in a patient to create an access path for a medical procedure. The medical procedure can then be performed through working cannula 140 using additional tools 150 (e.g., needle/stylet, drill, curette, facet/pedicle screw driver, an inflatable bone tamp, and/or a cement delivery tool). Instructions for use 160 provide guidance as to how to use the instruments in system 190, and can include directions for performing any of the techniques described herein or alternatives.

Introducer 130 includes an elongate shaft 131, a handle 132, a distal tip 134, and a longitudinal lumen 133. Tip 134 of introducer 130 is tapered (e.g., conical, bullet-shaped, rounded, or any other transition from a smaller to larger diameter) to allow shaft 131 to be more easily advanced through soft tissue.

Guide pin 120 is sized to fit within lumen 133 of introducer 130. Guide pin 120 further includes a marker element 121 at a proximal portion of guide pin 120, and introducer 130 further includes an indicator element 135 at a proximal portion of introducer 130. As described in greater detail below, marker element 121 and indicator element 135 cooperatively enable rapid and accurate determination of the extension of guide pin 120 beyond tip 134, without the need for additional measurement instruments. This extension (depth) measurement can then be used to select appropriately-sized tools and/or medical devices for subsequent use in the procedure being performed.

Note that for exemplary purposes, marker element 121 on guide pin 120 is depicted as a single dark mark or band. However, in various other embodiments, marker element 121 can take any form that permits positional information to be determined via indicator element 135 on introducer 130. For example, marker element 121 can include multiple markings, either evenly spaced or unevenly spaced. Alternatively, marker element 121 could include one or more features, such as grooves or raised elements in guide pin 120, or could even include one or more alternative materials at specific locations.

Similarly, indicator element 135 can take a variety of forms and constructions. For exemplary purposes, indicator element 135 is depicted as a ruler-type feature that includes a series tick marks adjacent to a window to allow the position of marker element 121 to be seen relative to the tick marks. In such an embodiment, the window of indicator element 135 (and in one embodiment, the entire handle 132) could be made out of a clear material, or the window could simply be an opening in introducer 130.

In various other embodiments, indicator element 135 could be a displacement sensor (e.g., linear encoder, hall effect sensor, LVDT, mechanical counter, etc.), proximity sensor (e.g., a magnetic, inductive, optical, or reflective sensor), or any other type of element responsive to (i.e., capable of detecting and/or tracking) marker element 121 on guide pin 120.

Note further that indicator element 135 is depicted as being located within handle 132 of introducer 135 for exemplary purposes only. In various other embodiments, indicator element 135 could be located on shaft 131 (rendering handle 132 an optional element of introducer 130) or on the surface of handle 132. In various other embodiments, handle 132 can be removable or even eliminated from introducer 130.

### Usage

FIGS. 1B through 1E show an exemplary use of system 190 to perform a medical procedure. In FIG. 1B, guide pin 120 is placed within a subject 100 into an interior anatomical target 110. In general, target 110 will be a bony or otherwise relatively hard internal body structure (e.g., a vertebra), although any identifiable anatomical target could be selected. By penetrating into the interior of target 110, guide pin 120 establishes a target depth for subsequent aspects of the procedure being performed on subject 100.

Working cannula 140 is slipped over shaft 131 of introducer 130 and is optionally interlocked with handle 132 to prevent relative rotation. Then, as shown in FIG. 1C, the assembly slides over guide pin 120 (guide pin 120 is inserted into lumen 133 of introducer 130). As introducer 130 contacts subject 100, tapered tip 134 allows introducer shaft 131 (and working cannula 140) to be pushed through the soft tissue of subject 100 until tip 134 contacts internal anatomical target 110, as shown in FIG. 1D.

At this point, the distance D between tip 134 of introducer 130 and the distal tip 122 of guide pin 120 is a key sizing parameter for subsequent steps in the medical procedure. For example, in certain posterior fixation procedures, the pedicles or facets of adjacent vertebrae are rigidly connected using screws, rods, plates or other structures to minimize relative motion of the vertebrae and thereby reduce pain caused by such relative motion. Thus, if target 110 represents a pair of adjacent vertebrae to be fixed in a posterior fixation to aid spinal fusion procedure, distance D could be indicative of the length of bone screw required to properly provide fixation of vertebrae across a facet joint (i.e., the thread engagement length necessary to ensure secure fastening without excessive vertebral penetration).

Accordingly, accurate determination of distance D can be critical to ensuring a successful patient outcome. By providing marker element 121 on guide pin 120, and corresponding indicator element 135 on introducer 130, the distance D can be read directly from the portion of introducer 130 external to subject 100. In the embodiment shown in FIGS. 1A-1D, distance D can be directly read from the alignment of marker element 121 and the corresponding tick mark D on indicator element 135.

Once distance D is determined from indicator element 135 and marker 121, introducer 130 and guide pin 120 can be removed from subject 100, leaving working cannula 140 in place. Optionally, working cannula 140 can be pushed into contact with target 110. In this manner, working cannula 140 provides an access path to target 110 for subsequent steps in the medical procedure. These subsequent steps can then be performed using tools (e.g., optional tools 150 of system 190) and/or devices selected based on the determination of distance D depicted in FIG. 1D.

Note that although FIGS. 1B-1E depict an exemplary process in which guide pin 120 is placed in subject 100 prior to placement of introducer 130, the placements of guide pin 120 and introducer 130 can take any order, or can even be performed concurrently. In various embodiments, any appropriate technique can be used to place guide pin 120 and introducer 130.

For example, FIGS. 1F-1I depict a common guide pin placement procedure using a needle 170 and a stylet 171. Needle 170 is a cannulated needle (typically 11-gauge) in which stylet 171 is slidably disposed. Needle 170 is deployed into the interior of subject 100 until it reaches internal anatomical target 110, as shown in FIG. 1F. At this point, stylet 171 is withdrawn, leaving behind needle 170, as shown in FIG. 1G. Guide pin 120 then slides into needle 170 as shown in FIG. 1H, and is driven into target 110 to the desired depth, typically by clamping a rotating chuck onto guide pin 120. Needle 170 is then removed, and introducer placement is performed as described with respect to FIGS. 1B-1D.

FIGS. 1J-1L depict an alternative guide pin/introducer placement procedure that does not make use of an initial needle deployment. Instead, as shown in FIG. 1J, introducer 130 (along with cannula 140) is plunged directly into subject 100 until introducer tip 134 contacts target 110. Optionally, a filler stylet 175 can be used to close lumen 133 to prevent debris entry into introducer 130. Stylet 175 is then removed from introducer 130 as shown in FIG. 1J, and guide pin 120 is driven into target 110 to a desired depth via lumen 133. The procedure can then continue as described with respect to FIGS. 1D-1E.

In another embodiment, stylet 175 in FIGS. 1J and 1K could be replaced with guide pin 120, thereby allowing introducer 130 and guide pin 120 to be placed concurrently. Various other approaches will be readily apparent.

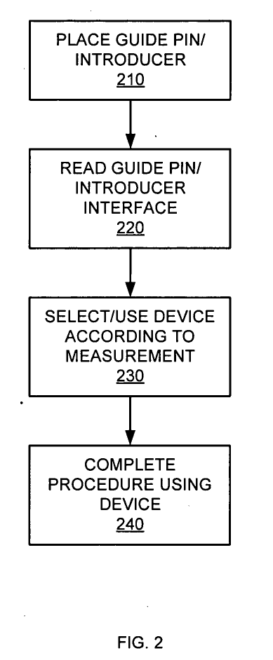

FIG. 2 shows a flow diagram of a process for performing a medical procedure using the system of FIG. 1A. In a PLACE GUIDE PIN/INTRODUCER step 210, guide pin 120 and introducer 130 (optionally with cannula 140) are placed within subject 100 such that tip 134 of introducer 130 is docked onto internal anatomical target 110 and distal tip 122 of guide pin 120 is placed a desired depth into target 110.

As described above with respect to FIGS. 1B-1D and 1F-1L, the placement order for guide pin 120 and introducer 130 can vary. For example, in one embodiment, guide pin 120 can be placed initially within target 110 and act as a guide for subsequent placement of introducer 130. In another embodiment, introducer 130 can be placed first and serve as a guide for subsequent placement of guide pin 120. In another embodiment, introducer 130 and guide pin 120 can be deployed as a unit into subject 100.

In any event, once guide pin 120 and introducer 130 are placed within subject 100, an extension depth D of distal tip 122 of guide pin 120 beyond tip 134 of introducer 130 is measured in a READ GUIDE PIN/INTRODUCER INTERFACE step 220. Specifically, as described with respect to FIG. 1D, the interaction between marker element 121 on guide pin 120 and indicator element 135 on introducer 130 is used to determine the distance D that guide pin 120 extends beyond tip 134 of introducer 130.

As noted above, while a simple graphical system (single marker on guide pin 120 aligned with tick marks on introducer 130) is depicted and described with respect to FIGS. 1A-1D for explanatory purposes, the interaction between marker element 121 and indicator element 135 can take any form that provides a measurement indication. For example, marker element 121 could be a series of markings, physical features, or material changes on guide pin 120 that are read by an optical, mechanical, electrical, magnetic, capacitive, or any other type of sensor in indicator element 135. Alternatively, marker element 121 could be a magnetic section of guide pin 120 providing a positional indication readable by a magnetic transducer in indicator element 135. Various other embodiments will be apparent.

Then, in a SELECT/USE DEVICE ACCORDING TO MEASUREMENT step 230, a tool, implant, device, or any other element of the medical procedure is selected and/or used based on the measurement taken in step 220. The procedure is then carried out in a COMPLETE PROCEDURE USING DEVICE step 240. Various procedural uses of the measurement information of step 220 will be readily apparent.

For example, a particular depth D measured in step 220 could indicate that a pedicle or facet screw of a particular length/size would be required for proper fixation in a spinal fusion process. Alternatively, a particular depth D could indicate that a vertebral compression fracture would be best treated by performing a kyphoplasty procedure using an inflatable bone tamp of a particular size, or by performing a vertebroplasty procedure in which the cement delivery nozzle is placed at a particular location within the vertebra. Various other procedural uses of the measurement information of step 220 will be readily apparent.

### Applications

In one embodiment, system 190 described with respect to FIGS. 1A-1L can be a system for percutaneous delivery of bone screws. Such a system can be used, for example, in posterior fixation to aid spinal fusion procedures to deliver facet screws or pedicle screws.

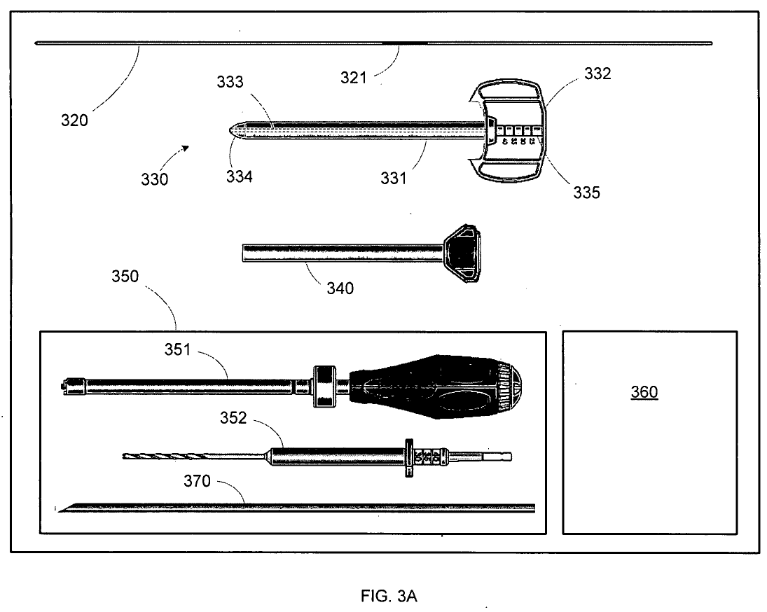

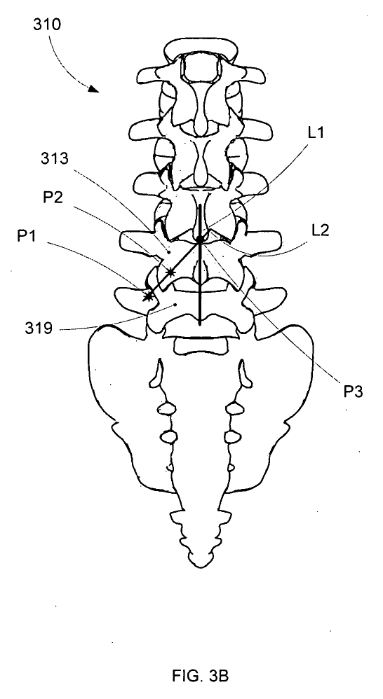

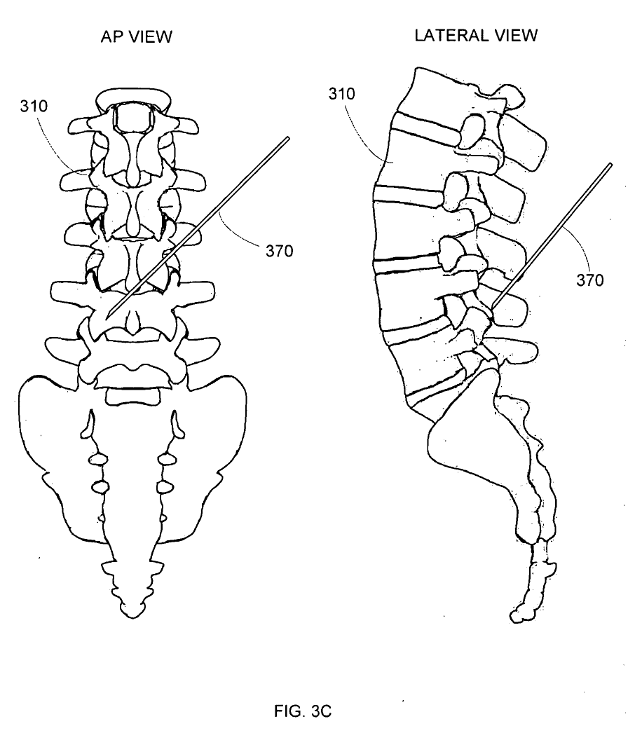

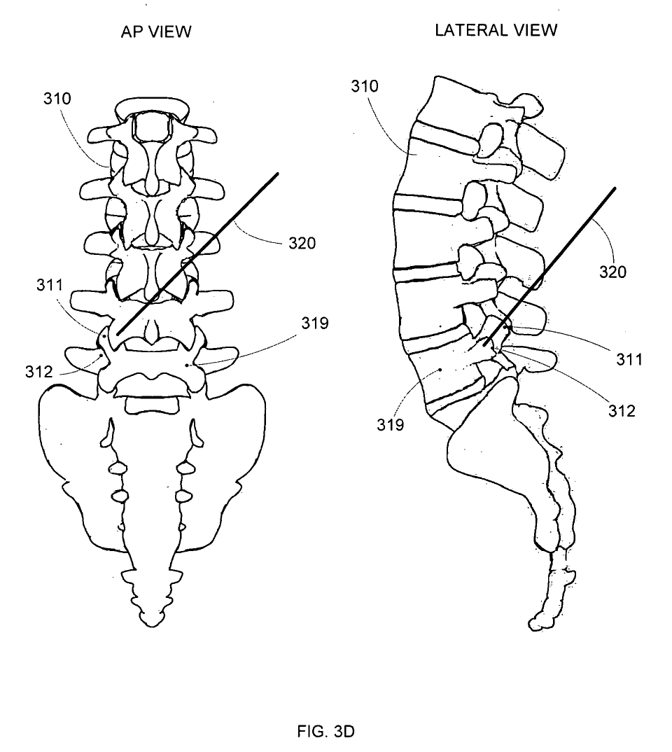

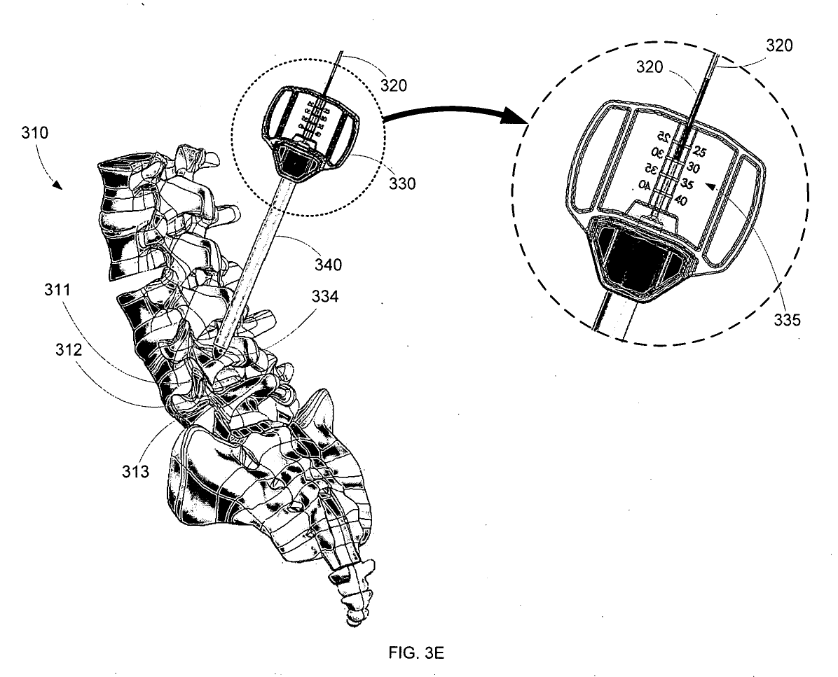

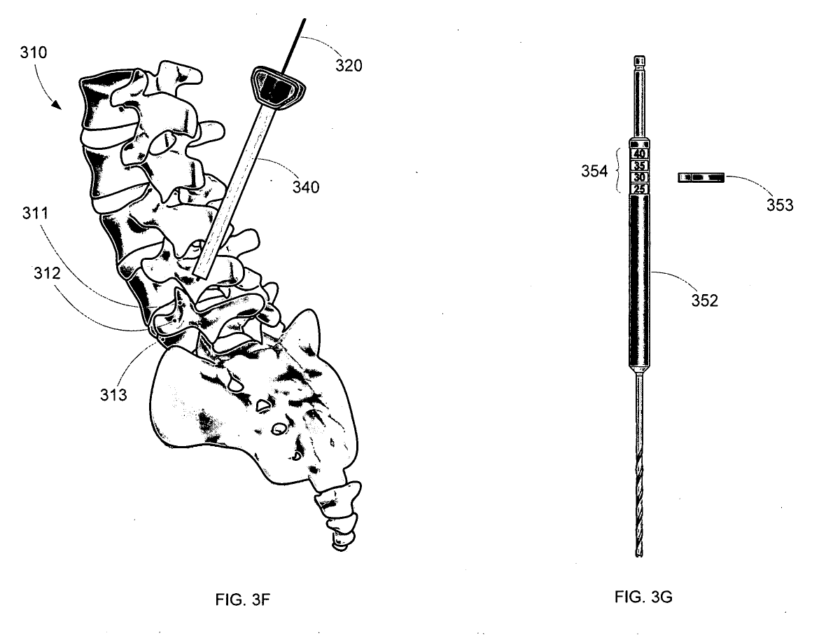

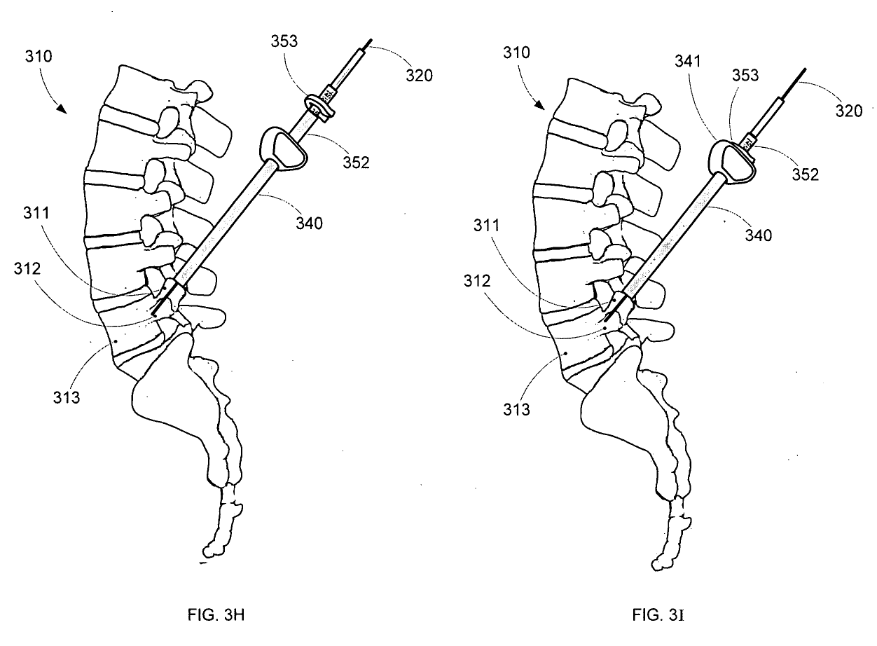

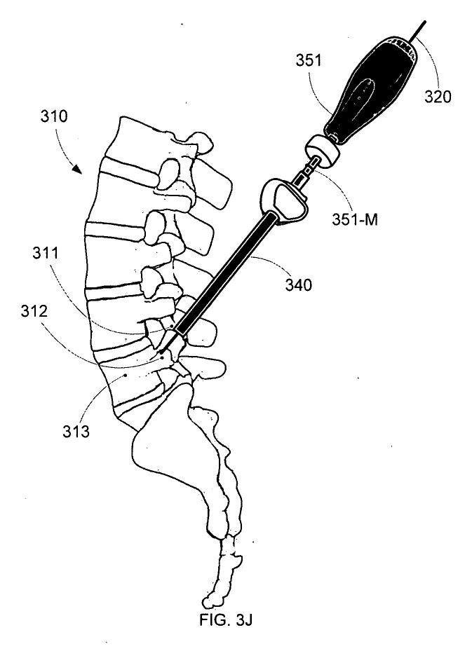

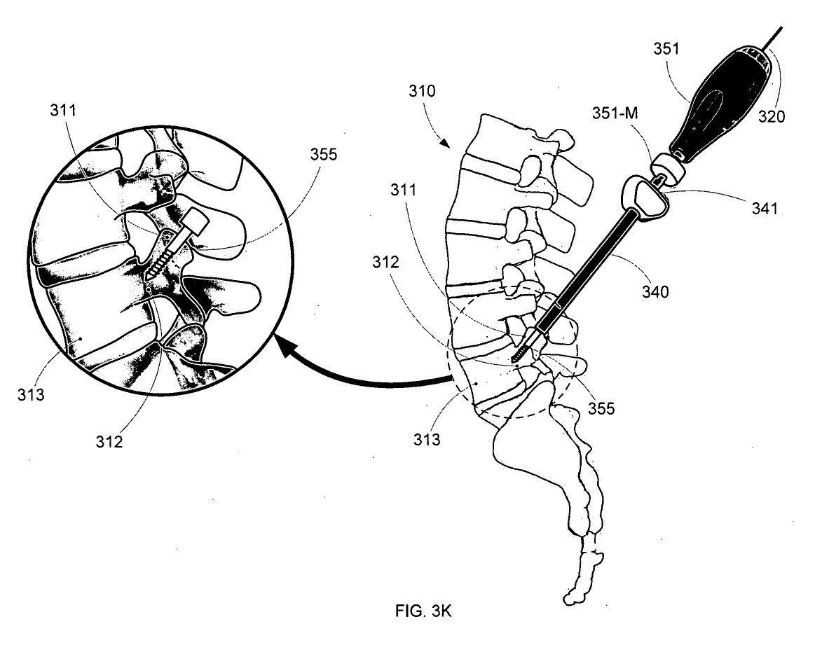

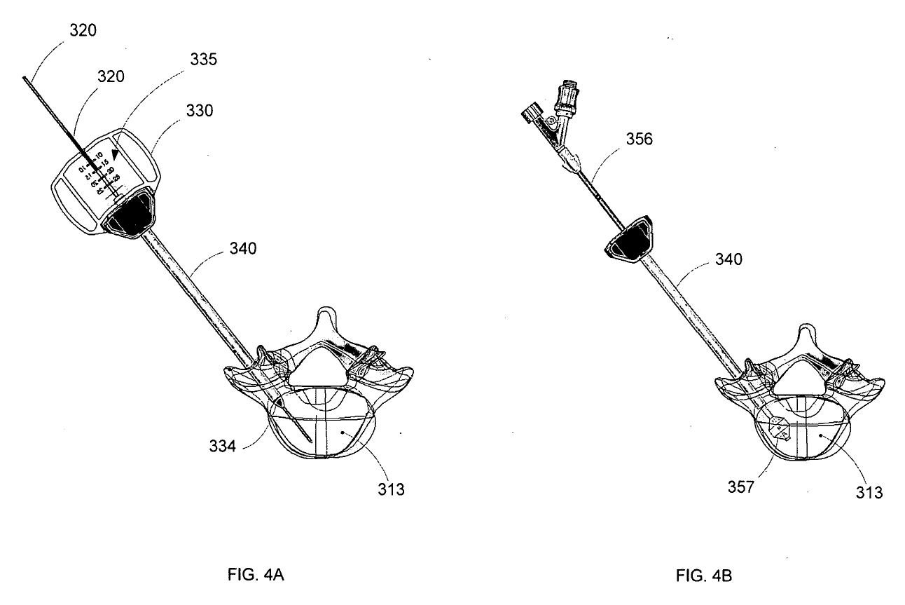

One such exemplary bone screw delivery system 390 is shown in FIG. 3A. System 390 includes a guide pin 320, an introducer 330, a working cannula 340, optional additional tools 350, and optional instructions for use 360. As noted above with respect to system 190, system 390 can be provided as a prepackaged kit as sterile, wrapped assembly, or the functional instruments of system 390 can be provided individually or in various combinations. Additional tools 350 include a cannulated needle 370 (with internal stylet 371 (not shown), a bone screw driver 351 and a cannulated drill bit 352, which are described in greater detail below.

In one embodiment, a posterior fixation procedure can be performed via transpedicular access. Specifically, the procedure can involve the posterior fixation of the two lateral facet joints of a single vertebra through a single percutaneous access point (“transpedicular access”). For example, FIG. 3B shows an Anterior-Posterior (AP) view of a spine 310 on which a posterior fixation procedure is to be performed between vertebrae 313 and 319.

To identify an appropriate percutaneous access point, the patient can be positioned such that the superior endplate of the inferior vertebra 319 has the appearance of a single line. A skin scribe can then be made along the mid pedicle and lateral border (point P1) and the medial border of the pedicle and superior endplate of the inferior vertebra 319 (point P2). A line L2 can then be drawn through points P1 and P2. The intersection point P3 of line L2 and the body (spine) midline L1 indicates the appropriate skin incision location for percutaneous access.

In this manner, access can be provided that minimizes the number of incisions required for a posterior fixation procedure. However, various other approaches will be readily apparent, and the techniques described herein are not limited to any particular access path(s) or procedure.

Once an access point has been selected, the procedure using system 390 (in this case a posterior fixation involving immobilizing adjacent vertebrae through the facet joint), cannulated needle 370 can be docked onto the desired location on a spine 310, as shown in FIG. 3C in both AP and lateral views. Stylet 371 can then removed from over stylet 371, and guide pin 320 can be inserted into needle 370 and driven across the facet joint 311 and into the pedicle 312 of the inferior vertebra 319, as shown in the AP and lateral views of FIG. 3D.

Introducer 330 can then be inserted over guide pin 320 (after removal of needle 370) and pushed through soft tissue (not shown for clarity) until tip 334 makes contact with the entry point on spine 310, as shown in FIG. 3E. As shown in the enlarged detail in FIG. 3E, the extension of guide pin 320 beyond tip 334 of introducer 330 can then be read from the interplay between marker element 321 on guide pin 320 and indicator element 335 on introducer 330.

In the example shown, the alignment between marker element 321 and the tick mark 30 on indicator element 335 represents the depth of the tip of guide pin 320 relative to the surface entry point on spine 310. Therefore, a facet screw having an appropriate length can be selected based on this measured depth. For example, in one embodiment, each tick mark of indicator element 335 corresponds to a specific bone screw size, and the tick mark closest to marker element 321 determines the screw size selection.

In this manner, guide pin 320 and introducer 330 not only define the trajectory and target location for the eventual placement of a facet screw in spine 310, but also provide accurate sizing information that can be used to ensure appropriate facet screw selection and installation, as described in greater detail below.

Next, as shown in FIG. 3F, introducer 330 can be removed, and working cannula 340 can be pushed towards spine 310 until it makes contact with spine 310 around the surface entry point defined by guide pin 320. Using the measurement previously taken using guide pin 320 and introducer 330, a spacer clip 353 can be attached to a corresponding depth marker 354 on cannulated drill bit 352, as shown in FIG. 3G.

Drill bit 352 can then be inserted over guide pin 320 and into cannula 340 to drill across facet joint 311 and into pedicle 312 until spacer clip 353 makes contact with the proximal end of cannula 340, as shown in FIGS. 3H and 3I. Because the length of cannula 340 is known, spacer clip 353 can act as a depth stop to ensure that the depth of the screw hole created by drill bit 352 is correctly sized for the selected facet screw. Note that although in various embodiments, drill bit 352 could be visually aligned with the proximal end of cannula 340, providing spacer clip 353 as a removable drill stop can more readily insure that drilling is performed to an accurate depth.

Drill bit 352 can then be removed and a cannulated screw 355 and screw driver 351 can be inserted over guide pin 320, as shown in FIG. 3J. Screw 355 can be self-tapped across facet joint 311 and into pedicle 312 by driver 351 as shown in FIG. 3K. In one embodiment, driver 351 can include a marker band 351-M that, when aligned with the proximal tip 341 of working cannula 340, indicates that screw 355 is substantially placed within the target region of spine 310.

Then, after any final fastening steps have been completed (e.g., facet screw nut tightening, retention feature deployment, or any other post-delivery action), driver 351, guide pin 320, and cannula 340 can be removed from the subject, leaving facet screw 355 fully and accurately placed within spine 310, as shown in the magnified detail in FIG. 3K. The process can then be repeated for other facet joints as required for proper inter-vertebral fixation. If the percutaneous access point has been selected as described with respect to FIG. 3B, a facet screw can be deployed in the adjacent lateral facet joint of vertebra 313 through that same percutaneous access point (i.e., transpedicular access) by repeating the procedure described with respect to FIGS. 3C-3K.

In another embodiment, system 190 described with respect to FIGS. 1A-1L can be a system for treatment of vertebral compression fractures (VCFs). Such a system can be used, for example, in vertebroplasty or kyphoplasty procedures to perform void creation (kyphoplasty) and/or cement delivery (vertebroplasty and kyphoplasty) within a vertebra. In such a case, bone screw driver 351 in system 390 shown in FIG. 3A could be replaced with one or more inflatable bone tamps (IBTs) or other void creation devices and/or cement delivery devices.

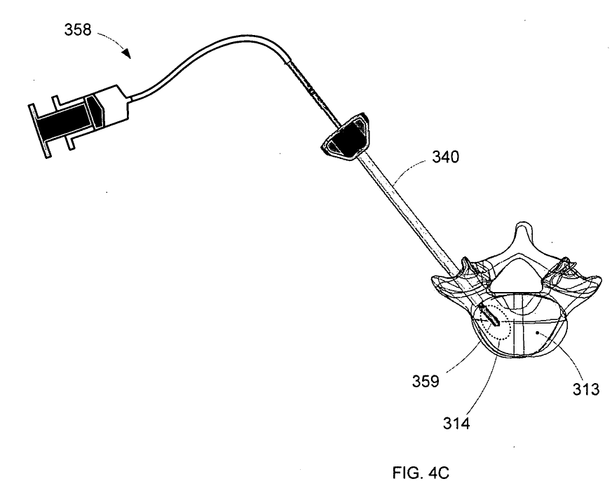

The use of such a system would begin with the placing of guide pin 320 and introducer 330 (along with cannula 340) at a target vertebra 313, as shown in FIG. 4A. The actual placement of guide pin 320 and introducer 330 could be performed as described above with respect to FIGS. 1J-1L or FIGS. 3A-3F. A depth measurement for guide pin 320 beyond tip 334 of introducer 330 could then be determined based on the interplay between marker element 320 and indicator element 335, as described with respect to FIG. 3F.

The resulting measurement could then be used to select an appropriately sized IBT 356 as shown in FIG. 4B. Specifically, an IBT 356 having a balloon 357 that is neither too long nor too short to create a properly placed void within vertebra 313 can be selected based on the measurement taken using guide pin 320 and introducer 330.

Additionally or alternatively, the measurement taken using guide pin 320 and introducer 330 could be used delivery of bone filler material (e.g., bone cement) to a kyphoplasty, vertebroplasty, or any other bone target. For example, FIG. 4C shows an exemplary cement delivery system 358 that includes a cement delivery nozzle 359 to provide filler material to a cement delivery target region 314 in vertebra 313.

In a kyphoplasty procedure, target region 314 can be the void created by IBT 356 shown in FIG. 4B. Using the depth measurement taken as described with respect to FIG. 4A, cement delivery nozzle 359 can be placed at the midpoint of target region 314 (or any other position within target region 314) to provide an even fill of target region 314.

In a vertebroplasty procedure, target region 314 can be the region spanned by the extension of guide pin 320 into vertebra 313 as described with respect to FIG. 4A. The depth measurement could then allow cement delivery nozzle 359 to be placed anywhere along that target region based on cement characteristics, structural characteristics of vertebra 313, or any other desired parameter(s).

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

1. A system comprising:
an introducer for creating a passage through tissue, the introducer comprising an elongate shaft having a distal tip, an introducer lumen through the elongate shaft, and an indicator element; and
a guide pin sized to pass through the introducer lumen, the guide pin comprising a marker element,
wherein when the guide pin is within the introducer lumen of the introducer and extended beyond the distal tip by a first distance, the indicator element in cooperation with the marker element indicates the distance.

2. The system of claim 1, wherein the marker element comprises at least one mark on the guide pin, and
wherein the indicator element comprises:
a window to the introducer lumen; and
a series of tick marks adjacent to the window.

3. The system of claim 2, wherein the introducer further comprises a handle connected to a proximal portion of the elongate shaft,
wherein the introducer lumen extends through the handle, and
wherein the handle comprises the indicator element.

4. The system of claim 3, further comprising a bone screw driver tool, and
wherein each of the series of tick marks corresponds to one of a plurality of bone screw lengths.

5. The system of claim 4, further comprising:
a cannula having a working lumen sized to fit over the elongate shaft;
a cannulated drill bit sized to fit through the working lumen and having a drill lumen sized to accommodate the guide pin; and
wherein the cannulated drill bit comprises a set of markings,
wherein each of the set of markings corresponds to one of the plurality bone screw lengths, and
wherein when one of the set of markings is aligned with a proximal end of the cannula, a distal end of the cannulated drill bit extends beyond a distal end of the cannula by a corresponding one of the plurality of bone screw lengths.

6. The system of claim 5, further comprising a removable drill stop, the removable drill stop being securable to the cannulated drill bit at each of the set of markings,
wherein when the removable drill bit is secured to the cannulated drill bit at one of the set of markings, the cannulated drill bit is prevented from entering the proximal end of the cannula beyond the one of the set of markings

7. The system of claim 3, further comprising a plurality of void creation tools having different sizes of void creation elements, and
wherein each of the series of tick marks corresponds to one of the sizes of void creation elements.

8. The system of claim 7, wherein the plurality of void creation tools comprises a plurality of inflatable bone tamps, and
wherein the void creation elements comprise balloons.

9. The system of claim 1, further comprising:
a cannulated needle having a needle lumen; and
a stylet sized to fit within the needle lumen,
wherein the guide pin is sized to fit within the needle lumen.

10. The system of claim 1, further comprising a stylet sized to removably fit within and substantially fill the introducer lumen.

11. The system of claim 1, wherein the indicator element comprises at least one of a displacement sensor and a proximity sensor responsive to the marker element.

12. A method for performing a medical procedure, the method comprising:
placing a guide pin at a target location in a body;
creating a passageway to the target location using an introducer, wherein the guide pin is positioned within an internal lumen of the introducer; and
determining a distance the guide pin extends beyond a distal end of the introducer based on at least one marking on a proximal portion of the guide pin and an indicator element on a proximal portion of the introducer.

13. The method of claim 12, wherein the indicator element comprises a window to the internal lumen of the introducer and a series of tick marks adjacent to the window, and
wherein determining the distance comprises comparing a position of the at least one marking relative to the series of tick marks.

14. The method of claim 13, further comprising:
identifying one of the series of tick marks closest to the at least one marking;
selecting a bone screw having a length corresponding to the identified one of the series of tick marks; and
deploying the bone screw at the target location through the passageway.

15. The method of claim 14, wherein deploying the bone screw comprises:
drilling a pilot hole at the target location to a depth equal to the length; and
screwing the bone screw into the pilot hole

16. The method of claim 15, wherein drilling the pilot hole comprises:
removing the introducer from a working cannula, the working cannula defining the passageway;
pushing the working cannula until a distal end of the working cannula contacts the target location;
positioning a drill stop on a drill bit;
placing the drill bit into the working cannula;
drilling into the target location until the drill stop contacts a proximal end of the working cannula, wherein when the drill stop contacts the proximal end of the working cannula, the drill bit is extended from the distal end of the working cannula by the length.

17. The method of claim 13, wherein the target location comprises an interior of a bone, the method further comprising:
identifying one of the series of tick marks closest to the at least one marking;
selecting an inflatable bone tamp with a balloon having a balloon length corresponding to the identified one of the series of tick marks;
removing the introducer and the guide pin from the passageway;
placing the balloon of the inflatable bone tamp into the target location;
inflating the balloon to create a void in the bone;
removing the inflatable bone tamp from the passageway; and
filling the void with bone filler material.

18. The method of claim 12, wherein the guide pin comprises a guide pin lumen,
wherein placing the guide pin at the target location comprises placing a cannulated needle with an internal stylet at the target location, removing the internal stylet from the cannulated needle, sliding the guide pin into the cannulated needle, driving the guide pin into the target location, and removing the cannulated needle, and
wherein creating the passageway comprises sliding the introducer lumen over the guide pin until the distal tip of the introducer contacts a surface of the target location.

19. The method of claim 12, wherein creating the passageway comprises filling the introducer lumen with a stylet and pushing the introducer into the body until the distal tip of the introducer contacts a surface of the target location, and
wherein placing the guide pin at the target location comprises removing the stylet from the introducer lumen, sliding the guide pin into the introducer lumen, and driving the guide pin into the target location.

20. The method of claim 14, wherein placing the guide pin comprises creating a percutanous access location on the body and introducing the guide pin into the body through the percutaneous access location,
wherein deploying the bone screw comprises screwing the bone screw through a facet joint of a superior vertebra into a pedicle of an inferior vertebra, the method further comprising:
placing a second guide pin at a second target location in the body by introducing the second guide pin into the body through the percutaneous access location;
creating a second passageway to the second target location using a second introducer, wherein the second guide pin is positioned within an internal lumen of the second introducer;
determining a distance the second guide pin extends beyond a distal end of the second introducer by comparing a position of at least one marking on the proximal portion of the second guide pin relative to a series of tick marks adjacent to a window to the internal lumen of the second introducer;
identifying one of the series of tick marks adjacent to the window to the internal lumen of the second introducer closest to the at least one marking on the proximal portion of the second guide pin;
selecting a second bone screw having a length corresponding to the identified one of the series of tick marks adjacent to the window to the internal lumen of the second introducer; and
deploying the second bone screw at the second target location through the second passageway by screwing the second bone screw through a second facet joint of the superior vertebra into a second pedicle of the inferior vertebra.
